(19) United States
(12) Patent Application Publication
(10) Pub. No.: US 2022/0062217 A1
(43) Pub. Date: Mar. 3, 2022

(54) PROPHYLAXIS AND MITIGATION OF MIGRAINE HEADACHES USING DIETARY PRECURSORS, PHOSPHOLIPIDS, MINERALS AND OTHER KETOGENIC SOURCES

(71) Applicant: Robert Firger, Bloomfield, CT (US)

(72) Inventor: Robert Firger, Bloomfield, CT (US)

(73) Assignee: Ultimate Brain Nutrients, Bonita Springs, FL (US)

(21) Appl. No.: 17/463,189

(22) Filed: Aug. 31, 2021

Related U.S. Application Data
(63) Continuation-in-part of application No. 17/011,650, filed on Sep. 3, 2020, now abandoned.
Application No. 17/011,650 is a continuation-in-part of application No. 16/501,502, filed on Apr. 22, 2019, now abandoned.
Application No. 16/501,502 is a division of application No. 15/743,448, filed on Jan. 10, 2018, now abandoned.

(51) Int. Cl. A61K 31/23; A61K 36/889; A61K 36/28; A61P 25/06

(52) U.S. Cl. CPC A61K 31/23; A61P 25/06; A61K 36/28; A61K 36/889

(57) ABSTRACT

A composition comprising at least one dietary precursor, at least one phospholipid, and at least one mineral, the composition is designed to reduce migraine symptoms in a patient experiencing migraine headaches.

### RELATED APPLICATIONS

This is a Continuation in part of U.S. application Ser. No. 17/011,650 entitled “Prophylaxis and Mitigation of Migraine Headache Using Medium Chain Triglycerides Ketone Esters, and Other Ketogenic Sources” filed on Sep. 3, 2020 which was a continuation in part of U.S. application Ser. No. 16/501,502 entitled “Prophylaxis and Mitigation of Migraine Headache Using Medium Chain Triglycerides Ketone Esters, and Other Ketogenic” a divisional of U.S. Application filed on April 22,2019, which is a divisional of U.S. application Ser. No. 15/743,448 entitled “Prophylaxis and Mitigation of Migraine Headache Using Medium Chain Triglycerides Ketone Esters, and Other Ketogenic Sources” filed on Jan. 10, 2018, which has now been granted a Notice of Allowance.

### FIELD OF THE INVENTION

The invention relates to migraine headaches, sometimes simply called migraine. More particularly, the invention relates to preventing, arresting, or reducing the frequency or severity of migraine.

### BACKGROUND OF THE INVENTION

Migraine is a neurological disease with a strong genetic component. It is characterized by episodes of disabling headache, often called migraine attacks. They are clinically quite different from regular headaches which are non-migrainous. There are about 100 million people with headaches in the U.S. and about 37 million of these people have migraines. The World Health Organization estimates that approximately 18 percent of Women and 7 percent of men in the U.S. suffer from migraines. People who suffer from migraine are known as “migraineurs”. Migraine ranks in the top 20 of the World's most disabling medical illnesses. (See Global Burden of Disease Study, updated 2004, World Health Organization)

Migraines are called primary headaches because the pain isn't caused by another disorder or disease such as a brain tumor or head injury. Some cause pain on just the right side or left side of the head (hemicrania), others result in generalized head pain. Migraine sufferers may have moderate or severe pain and usually can't participate in normal activities during the duration of the attack, because of the pain. Often when a migraine strikes, people can't think beyond trying to find a quiet, dark room. More than 90% of sufferers are unable to function normally during their migraine attacks. Because of migraine-induced disability, American employers lose more than $13 billion each year as a result of 113 million lost Work days.

Many people experience migraine attacks lasting for at least four hours and the maigraine may last for days. The range of time someone is affected by an attack is actually longer than the full-blown attack itself, as there is a pre-monitory, or build-up phase, and a post-dromal phase that can last one to two days. An estimated 14 million individuals in the U.S. are classified as “chronic migraneurs”, meaning that they suffer a migraine episode a minimum of 15 days each month. For all intents and purposes, this means that with migraine symptoms usually lasting multiple days, these sufferers may experience the impact of migraine on their lives virtually every day.

Recent research has highlighted the potential importance of neurogenic inflammation in migraine pathophysiology and pharmacology. Extravasation (inflammatory leakage of white blood cells from the capillaries), vasodilatation (widening of blood vessels resulting in lowered blood pressure), mast cell activation, and the release of pro-inflammatory mediators may activate trigeminal afferents (the nerve fibers of the fifth cranial nerve governing sensation in the face and certain facial motor activity), thus leading to sensitization in the migraineur. There are indications that blockading vasodilatation in inflammation may be effective in the treatment of migraine. It has been shown that the ketone body [3-hydroxybutyrate has beneficial effects on inflammation, with significant potential for addressing aspects of a variety of metabolic related diseases.

### Current Treatments of Migraine:

A number of different medications have been used to treat migraine, with varying levels of effectiveness. All of these treatments, however, with increased frequency of use can lead to a condition called medication overuse headache (MOH). Medication-overuse headaches occur when medications not only stop relieving pain but themselves also cause headaches. Patients then use more pain medication, which may lengthen the duration of pain of the migraine sufferer. In addition, these treatments pose additional, specific risks such as drug sensitivities, cognitive symptoms and negative physiological effects, some of which are further elucidated below.

For pain associated with mild or moderate migraine, aspirin or non-steroidal anti-inflammatory drugs (NSA1Ds, such as ibuprofen and naproxofen) are commonly used. Unfortunately, regular use of these medication. 3s can result in abdominal pain and intestinal ulcers-often associated with hemorrhage, and they are generally not effective against more severe migraine.

Front-line treatments for more severe migraine include the triptans, such as sumatriptan (Imitrex), rizatriptan (Maxalt), almotriptan (Axert), naratriptan (Amerge), zolmitriptan (Zomig), frovatriptan (Frova) and eletriptan (Relpax). In addition to MOH, these medications can cause a condition called “serotonin syndrome” when used together with selective serotonin reuptake inhibitors (SSRIs), including Prozac, Celexa, Luvox, Zoloft, Paxil and Lexapro; or serotonin and norepinephrine inhibitors (SNRIS), including Effexor, Pristiq and Cymbalta, all of which are commonly prescribed for depression and/or anxiety. These are mental health conditions for which migraneurs already may be receiving treatment with some frequency. The front-line medications, discussed above, are often serotonin agonists whose nature it is to raise serotonin levels. When taken in combination, they can cause serotonin levels in the CNS (central nervous system) to rise to especially high levels. “Serotonin syndrome” is a rare, potentially life-threatening condition that occurs when an excess of serotonin-a neurotransmitter of major importance in the central nervous system (CNS) accumulates in the brain.

Less effective than triptans are ergotamines, such as ergotamine and dihydroergotamine. Ergotamines appear to be most useful for people who have migraines that last a long period of time, but don't have frequent migraine attacks. Side effects of ergotamines include dizziness, nausea and vomiting, cold, clammy hands and feet, muscle pain, numbness, and feelings of discomfort or anxiety. Ergotamines also can have serious interactions with a large number of drugs.

Medications containing narcotics, particularly codeine, are sometimes used to treat migraine headache pain for people who can't take triptans or ergotamines. Narcotics are seriously habit-forming and are usually used only as a last resort.

Glucocorticoids, such as prednisone and dexamethasone may be used in conjunction with other medications to improve pain relief Because of the risk of adverse side effects, glucocorticoids should not be used frequently or for prolonged periods, which presents problems for chronic migraneurs.

Due to the severity and frequency of the side effects caused by many of these medications that treat symptoms of migraine, another strategy is to utilize medications that reduce the frequency of migraine attacks.

One front line treatment for reducing frequency of migraine onsets is beta blockers, which are normally and usually prescribed for high blood pressure and heart problems. Unfortunately, beta blockers produce their own, often severe side effects, including fatigue, nausea, reduced ability to exercise, insomnia, sleep problems, nightmares and vivid dreams, memory problems, depression, weight gain, and exacerbation of asthma.

Calcium channel blockers, which are also normally prescribed for high blood pressure and heart problems, are sometimes prescribed to reduce frequency of migraine onset. However, these drugs carry their own side effect profile, including dizziness, drowsiness, constipation, nausea, headache, rash, pitting edema of the feet, ankles and lower legs (owing to water retention), low blood pressure, tachycardia (rapid heartbeat), and flushing (reddening) of the face, neck and/or upper chest.

Tricyclic antidepressants are sometimes used to reduce frequency of migraine onset. Unfortunately, these drugs have a wide range of undesirable side effects, including disturbances in heart rhythm, increased sensitivity to sunlight, drowsiness, dry mouth, painful urination, sexual dysfunction, weight gain, dizziness, lightheadedness, and headaches. In addition, antidepressants come with the U.S. Food and Drug Administration's strongest “black box” warning that the use of antidepressants has been associated with an increased risk of suicidal thoughts and behaviors in children, adolescents and young adults. Tricyclic antidepressants can be fatal in overdose.

Anticonvulsants are sometimes prescribed to reduce frequency of migraine onset. Only Depakote (gen. valproic acid) and Topamax (gen. topiramate) have been approved for this purpose. However, some anticonvulsant medications may reduce the effectiveness of oral contraceptives, since their induction of the hepatic cytochrome P450 (CYP45 0) isoenzyme causes decreased sex hormone levels in women taking oral contraceptives, thus raising the potential for decreased effectiveness of oral contraceptives and increased risk of unplanned pregnancy. In addition, more than half of people who take anticonvulsant drugs report experiencing at least one side effect, the most common of which are dizziness, nausea and sleepiness. Some people who take newer drugs in this class also experience swelling in the feet and hands, weight gain, blurry vision, trouble concentrating, and lapses in memory.

More recently, injections of botulinum toxin (Botox®) have been used to reduce frequency of migraine onset. However, Botox's side effects include headache, facial loss of movement, eyelid drooping, lung inflammation, neck pain, muscle stiffness and weakness, muscle pain and spasms, pain at injection site, and high blood pressure. In addition, the paralyzing effect of Botox can spread to other areas of the body and can cause general weakness, double vision, difficulty swallowing, voice and speech disorders, loss of bladder control and difficulty breathing. Given the frequency and severity of side effects of both medications to treat symptoms of migraine and to reduce frequency of migraine onset, there clearly is a need for safer treatments to prevent or reduce frequency of migraine onset and reduce migraine symptoms. Although not yet a medically accepted treatment for migraine, some references have suggested the use of a ketogenic diet for this purpose (see Maggioni. F. et al. Cephalalgia 31. 1150-1). However, some authors have described ketogenic dieting as potentially causing severe headaches, and low glucose levels, among a number of other physiological effects of ketogenic dieting, which might complicate its use. In addition, adherence to a ketogenic diet can be difficult and compliance problems frequently arise.

In recent years, the FDA has approved newer drugs for people who suffer from frequent and debilitating migraines. These new drugs are very expensive and require injections for treatment of a patient with migraines. These drugs are designed to prevent migraines and to reduce the frequency, intensity and duration of migraines. These injectable drugs aim to address calcitonin gene-related peptide (CGRP), CGRP is found throughout the body and causes blood vessels to vasodilate, researches have long believed that vasodilation is one of the key contributors to migraine headaches. The new injectable drugs block CGRP, by either sticking onto CGRP or sitting on the receptors that receive CGRP. The four current injectable drug options are eptinezumab (Vyepti), erenumab (Aimoving), fremanezumab (Ajovy) and galcanezumab (Emgality).

### SUMMARY OF THE INVENTION

The invention provides effective, safer treatments to prevent or reduce frequency of migraine onset and reduce migraine symptoms. The present inventors have surprisingly discovered that providing ketogenic medium chain triglycerides (KMCT) to patients suffering from frequent migraine attacks can reduce frequency of migraine onset and reduce migraine symptoms, without the side effects of medications currently used for this purpose. KMCT are metabolized in the liver to provide a rich source of ketone bodies, which can be metabolized as a carbon and energy source for the body, especially the brain. Unlike ketogenic dieting, providing exogenous KMCT does not result in reduced glucose concentrations or other physiological effects associated with ketogenic dieting, and does not suffer from similar patient compliance issues. Ingestion of KMCT has no reported serious side effects and only minor and transitory reported effects of gastro-intestinal distress or sensitivity in some users, which has been shown to usually diminish with continued use.

In one embodiment, a composition comprising at least one dietary precursor, at least one phospholipid, and at least one mineral, said composition is designed to reduce migraine symptoms in a patient experiencing migraine headaches.

In another embodiment, a composition wherein the dietary precursor is a ketone precursor which is ketogenic MCT.

In still another embodiment, a composition of wherein the phospholipid is selected from a group consisting essentially of phosphatidylserine, phosphatidylcholine, phosphatidylinositol, phosphatidylethanolamine, phosphatidylglycerol and combinations thereof.

In yet another embodiment, a composition wherein at least one mineral is selected from a group comprising magnesium, calcium, zinc, phosphorus, potassium, chloride, iron, zinc, iodine, sulfur, cobalt, fluoride, manganese, selenium, chromium, and mixtures or combinations thereof. composition of claim 1 further comprising additives, colorant, flavorings and a carrier.

In still yet another embodiment, a composition of wherein the carrier is selected from a group consisting essentially of gel, powder, liquid, topical, tablets, gel caps, sprays, time release mechanism, pill, boosters, intravenous and mixtures and combinations thereof.

In a further embodiment, a composition comprising at least one dietary precursor, at least one phospholipid, at least one mineral, and at least one dietary ketone, the composition is designed to reduce migraine symptoms in a patient experiencing migraine headaches.

In still a further embodiment, a composition wherein the dietary precursor is a ketone precursor which is ketogenic MCT.

In still yet a further embodiment, a composition of wherein the phospholipid is selected from a group consisting essentially of phosphatidylserine, phosphatidylcholine, phosphatidylinositol, phosphatidylethanolamine, phosphatidylglycerol and combinations thereof.

In another embodiment, a composition wherein the at least one mineral is selected from a group comprising magnesium, calcium, zinc, phosphorus, potassium, chloride, iron, zinc, iodine, sulfur, cobalt, fluoride, manganese, selenium, chromium, and mixtures or combinations thereof.

In yet another embodiment, a composition of wherein the dietary ketone is selected from a group consisting essentially of ketones, ketone esters, ketone salts and combinations thereof.

In still yet another embodiment, a composition wherein the dietary ketone is selected from a group consisting essentially of 3-hydroxybutyrate-1, 3-butanediol monoester, glyceryl-tris-3-hydroxybutyrate, R-3-hydroxybutyrate-R-1,3-butanediol monoester and D-13-hydroxybutyrate-(R)-1,3-butanediol and mixtures and combinations thereof.

In a further embodiment, a composition wherein the dietary ketone is given to a patient in an amount sufficient to raise plasma ketone bodies to a level from about 0.5 mM to about 6 mM.

In still a further embodiment, a composition wherein the phospholipid is given to a patient in an amount from about 25 mg to about 2,000 mg.

In still yet a further embodiment, a composition further comprising at least one Vitamin B. In yet a further embodiment, a composition wherein the at least one Vitamin B is selected from a group comprising nicotinamide, niacinamide, niacin, nicotinic acid and mixtures or combinations thereof.

In another embodiment, a composition further comprising additives, colorant, flavoring and a carrier.

In yet another embodiment, a composition wherein the carrier is selected from a group consisting essentially of gel, powder, liquid, topical, tablets, gel caps, sprays, time release mechanism, pill, boosters, intravenous delivery system and mixtures and combinations thereof.

In a further embodiment, a method of manufacturing a composition for treatment of symptoms of migraines, said method comprises admixing at least one dietary precursor, at least one phospholipid, at least one mineral, additives, colorant, and flavoring with an carrier to form a composition designed to reduce migraine symptoms in a patient experiencing migraine headaches.

In still a further embodiment, a method further comprising admixing at least one ketone and at least one Vitamin B.

In a further embodiment, a composition comprising at least one dietary ketone, at least one phospholipid, at least one mineral, and at least one form of Vitamin B, the dietary ketone, the phospholipid, the mineral, and the Vitamin B are designed to reduce migraine symptoms in a patient experiencing migraines.

In another embodiment, a composition wherein said dietary ketone is selected from a group consisting essentially of KMCT, ketones, ketone esters, ketone salts and combinations thereof.

In yet another embodiment, a composition of wherein the dietary ketone is designed to prevent the onset of migraines in a patient with a history of migraines.

In a further embodiment, a composition wherein the dietary ketone is designed to treat the onset of migraines in a patient with a history of migraines.

In yet a further embodiment, a composition of wherein the dietary ketone is given to a patient in an amount sufficient to raise plasma ketone bodies to a level from about 0.5 mM to about 6 mM.

In still another embodiment, a composition of wherein the dietary ketone is selected from a group consisting essentially of 3-hydroxybutyrate-1, 3-butanediol monoester, glyceryl-tris-3-hydroxybutyrate, R-3-hydroxybutyrate-R-1, 3-butanediol monoester and D-13-hydroxybutyrate-(R)-1,3-butanediol and mixtures and combinations thereof.

In still yet another embodiment, a composition of wherein the dietary ketone is provided to a patient in an amount sufficient to reduce the symptoms of migraines.

In a further embodiment, a composition wherein the dietary ketone is provided to a patient in an amount sufficient to prevent the onset of migraines.

In still another embodiment, a composition wherein the dietary ketone is given to a patient in an amount sufficient to raise plasma ketone bodies to a level from about 2 mM to about 5 mM.

In another embodiment, a composition wherein the phospholipid is selected from a group consisting essentially of Phosphatidylserine, phosphatidylcholine, phosphatidylinositol, phosphatidylethanolamine, phosphatidylglycerol and combinations thereof.

In a further embodiment, a composition wherein the phospholipid is designed to prevent the onset of migraines in a patient with a history of migraines.

In still another embodiment, a composition wherein the phospholipid is given to a patient in an amount in an amount from about 25 mg to about 2,000 mg.

In yet another embodiment, a composition wherein the phospholipid is given to a patient in an amount of 100 mg.

In still yet another embodiment, a composition wherein the phospholipid is selected from a group consisting essentially of Phosphatidylserine, phosphatidylcholine, phosphatidylinositol, phosphatidylethanolamine, phosphatidylglycerol and combinations thereof.

In a further embodiment, a composition wherein the phospholipid is provided to a patient in an amount sufficient to reduce the symptoms of migraines.

In still a further embodiment, a composition wherein the dietary ketone is given to a patient in an amount sufficient to raise plasma ketone bodies to a level from about 0.5 mM to about 6 mM.

In yet a further embodiment, a composition wherein the at least one mineral is selected from a group comprising magnesium, calcium, zinc, phosphorus, selenium, chromium, and mixtures or combinations thereof.

In another embodiment, a composition wherein the at least one form of Vitamin B is selected from a group comprising nicotinamide, niacinamide, niacin, nicotinic acid and mixtures or combinations thereof.

In yet a further embodiment, a method of manufacturing a composition for treatment of migraines, the method comprises admixing at least one dietary ketone, at least one phospholipid, at least one mineral, and at least one form of Vitamin B with an agent to form a composition designed to reduce migraine symptoms in a patient experiencing migraines.

In another embodiment, a method wherein the dietary ketone is designed to prevent the onset of migraines in a patient with a history of migraines.

In still another embodiment, a method wherein the dietary ketone is designed to treat the onset of migraines in a patient with a history of migraines.

In a further embodiment, a method wherein the dietary ketone is selected from a group consisting essentially of KMCT, ketones, ketone esters, ketone salts and combinations thereof.

In still a further embodiment, a method wherein the dietary ketone is given to a patient in an amount sufficient to raise plasma ketone bodies to a level from about 2 mM to about 5 mM.

In still yet a further embodiment, a method wherein the dietary ketone is selected from a group consisting essentially of 3-hydroxybutyrate-1,3-butanediol monoester, glyceryl-tris-3-hydroxybutyrate, R-3-hydroxybutyrate-R-1,3-butanediol monoester and D-13-hydroxybutyrate-(R)-1,3-butanediol and mixtures and combinations thereof.

In another embodiment, a method wherein the agent is selected from a group consisting of pharmaceutical agents, non-pharmaceutical agents, stabilizing agents and mixtures and combinations thereof.

In yet another embodiment, a method wherein the phospholipid is designed to prevent the onset of migraines in a patient with a history of migraines.

In still another embodiment, a method wherein the phospholipid is selected from a group consisting essentially of Phosphatidylserine, phosphatidylcholine, phosphatidylinositol, phosphatidylethanolamine, phosphatidylglycerol and combinations thereof.

In a further embodiment, a method wherein the phospholipid is given to a patient in an amount in an amount from about 25 mg to about 2,000 mg.

In yet a further embodiment, a method wherein the phospholipid is given to a patient in an amount in an amount of 100 mg.

In another embodiment, a method wherein the dietary ketone is selected from a group consisting essentially of KMCT, ketones, ketone esters, ketone salts and combinations thereof.

In yet another embodiment, a method wherein the agent is selected from a group consisting of pharmaceutical agents, non-pharmaceutical agents, stabilizing agents and mixtures and combinations thereof.

In still another embodiment, a method wherein the at least one mineral is selected from a group comprising magnesium, calcium, zinc, phosphorus, selenium, chromium, and mixtures or combinations thereof.

In still yet another embodiment, a method wherein the at least one form of Vitamin B is selected from a group comprising nicotinamide, niacinamide, niacin, nicotinic acid and mixtures or combinations thereof.

In one embodiment of the invention provides a method for preventing or reducing the frequency of migraine onset in a migraineur. The method according to this aspect of the invention comprises providing the migraineur with dietary KMCT.

In another embodiment, the invention provides a method for reducing the migraine symptoms experienced by a migraineur. The method according to this aspect of the invention comprises providing the migraineur with dietary KMCT.

In yet another embodiment, the invention provides a method for augmenting the effects of pharmaceutical intervention to reduce the frequency of migraine onset in a migraineur. The method according to this aspect of the invention comprises providing the migraineur with dietary KMCT in combination with a pharmaceutical agent provided to reduce the frequency of migraine onset in a migraineur.

In still another embodiment, the invention provides a method for augmenting the effects of pharmaceutical intervention to reduce the migraine symptoms experienced by a migraineur. The method according to this aspect of the invention comprises providing the migraineur with dietary KMCT in combination with a pharmaceutical agent provided to reduce migraine symptoms experienced by a migraineur.

In a further embodiment, the invention provides a method for augmenting the effects of non-pharmaceutical intervention to reduce the frequency of migraine onset in a migraineur. The method according to this aspect of the invention comprises providing the migraineur with dietary KMCT in combination with a non-pharmaceutical intervention or agent.

In another further embodiment, the invention provides a method for augmenting the effects of non-pharmaceutical intervention to reduce migraine symptoms experienced by a migraineur. The method according to this aspect of the invention comprises providing the migraineur with dietary KMCT in combination with a non-pharmaceutical intervention or agent.

In yet another further embodiment, the invention provides a method for preventing or reducing the frequency of onset of migraine, and/or reducing migraine symptoms, comprising providing a migraineur with dietary ketone esters, ketone salts and other sources of ketone bodies.

In another embodiment, a composition comprising a dietary ketone, the dietary ketone is designed to reduce migraine symptoms in a patient experiencing migraines.

In a further embodiment, the dietary ketone is selected from a group consisting essentially of ketones, ketone esters, ketone salts and combinations thereof.

In yet another embodiment, the dietary ketone is designed to prevent the onset of migraines in a patient with a history of migraines.

In still even another embodiment, the dietary ketone is given to a patient in an amount sufficient to raise plasma ketone bodies to a level from about 2 mM to about 5 mM.

In still another embodiment, the dietary ketone is selected from a group consisting essentially of 3-hydroxybutyrate-1,3-butanediol monoester, glyceryl-tris-3-hydroxybutyrate, R-3-hydroxybutyrate-R-1, 3-butanediol monoester and D-13-hydroxybutyrate-(R)-1,3-butanediol and mixtures and combinations thereof.

In yet another embodiment, the dietary ketone is provided to a patient in an amount sufficient to reduce the symptoms of migraines.

In still yet another embodiment, the dietary ketone is provided to a patient in an amount sufficient to prevent the onset of migraines.

In still another further embodiment, the present invention provides for a method of reducing migraine symptoms, the method comprises providing a patient with dietary ketones designed to reduce migraine symptoms in a patient experiencing migraines.

In yet another further embodiment, the dietary ketone is selected from a group consisting essentially of ketones, ketone esters, ketone salts and combinations thereof.

In still yet another further embodiment, the dietary ketone is designed to prevent the onset of migraines in a patient with a history of migraines.

In yet another embodiment, the present invention provides for a method of manufacturing a composition for treatment of migraines, the method comprises admixing a dietary ketone with an agent to form a composition designed to reduce migraine symptoms in a patient experiencing migraines.

In still another embodiment, the dietary ketone is designed to prevent the onset of migraines in a patient with a history of migraines.

In yet another embodiment, the agent is selected from a group consisting of pharmaceutical agents, non-pharmaceutical agents, stabilizing agents and mixtures and combinations thereof.

### DETAILED DESCRIPTION OF THE INVENTION

As required, detailed embodiments of the present invention are disclosed herein; however, it is to be understood that the disclosed embodiments are merely exemplary of the invention that may be embodied in various forms. Therefore, specific structural and functional details disclosed herein are not to be interpreted as limiting, but merely as a basis for the claims and as a representative basis for teaching one skilled in the art to variously employ the present invention.

The specific examples below will enable the invention to be better understood. However, they are given merely by way of guidance and do not imply any limitation.

The invention relates to migraine headaches, sometimes simply called migraine. More particularly, the invention relates to preventing or reducing the frequency of migraine onset and reducing migraine symptoms. The invention provides effective, safer treatments to reduce frequency of migraine onset. The present inventors have surprisingly discovered that providing ketogenic medium chain triglycerides (KMCT) to patients suffering from frequent migraine attacks can reduce frequency of migraine onset and reduce migraine symptoms, without the side effects of medications currently used for this purpose. KMCT are metabolized in the liver to provide a rich source of ketone bodies, which can be metabolized as a carbon and energy source for the body, especially the brain. Unlike ketogenic dieting, providing exogenous KMCT does not result in reduced glucose concentrations or other physiological effects associated with ketogenic dieting, and does not suffer from similar patient compliance issues.

In one embodiment, the present invention provides for a method for preventing or reducing the frequency of migraine onset in a migraineur. The method according to this aspect of the invention comprises providing the migraineur with dietary KMCT. In another embodiment, the KMCT comprise a mixture of capric and caprylic triglycerides. In yet another embodiment, the KMCT are enriched or purified from coconut oil or palm oil. In still another embodiment, the KMCT comprise a mixture of capric and caprylic triglycerides and coconut oil or palm oil. In a further embodiment, the total KMCT provided to the migraineur is from about 3 g to about 30 g/day. In another further embodiment, the total KMCT provided to the migraineur is from about 3 g to about 15 g/day.

In a further embodiment, the invention provides a method for reducing the migraine symptoms experienced by a migraineur. The method according to this aspect of the invention comprises providing the migraineur with dietary KMCT. In some embodiments, the KMCT comprise a mixture of capric and caprylic triglycerides. In some embodiments, the KMCT are enriched or purified from coconut oil or palm oil. In some embodiments, the KMCT comprise a mixture of capric and caprylic triglycerides and coconut oil or palm oil. In some embodiments, the total KMCT provided to the migraineur is from about 3 g to about 30 g/day. In some embodiments, the total KMCT provided to the migraineur is from about 10 g to about 30 g/day.

A convenient source for such KMCT is Fuel for Thought® (Ultimate Brain Nutrients) an orally bioavailable nutritional supplement comprising capric and caprylic triglycerides and coconut oil. Other sources of KMCT, for example, are disclosed in co-pending US Applications Nos. 62/151,678 and 62/151,691.

In another further embodiment, the invention provides for a method for augmenting the effects of pharmaceutical intervention to reduce the frequency of migraine onset in a migraineur. The method according to this aspect of the invention comprises providing the migraineur with dietary KMCT in combination with a pharmaceutical agent provided to reduce the frequency of migraine onset in a migraineur. Such augmentation of pharmaceutical intervention may reduce the dosage and/or frequency of pharmaceutical agent required, thereby reducing unwanted side effects caused by the pharmaceutical agent. In some embodiments, the KMCT comprise a mixture of capric and caprylic triglycerides. In some embodiments, the KMCT are enriched or purified from coconut oil or palm oil. In some embodiments, the KMCT comprise a mixture of capric and caprylic triglycerides and coconut oil or palm oil. In some embodiments, the total KMCT provided to the migraineur is from about 3 g to about 30 g/day. In some embodiments, the total KMCT provided to the migraineur is from about 3 g to about 15 g/day. Pharmaceutical agents useful in this aspect of the invention include, without limitation, one or more of beta blockers, calcium channel blockers, tricyclic antidepressants, anticonvulsants (such as Depakote and Topamax), and Botox.

In a further embodiment, the invention provides a method for augmenting the effects of pharmaceutical intervention to reduce the migraine symptoms experienced by a migraineur. The method according to this aspect of the invention comprises providing the migraineur with dietary KMCT in combination with a pharmaceutical agent provided to reduce migraine symptoms experienced by a migraineur. Such augmentation of pharmaceutical intervention may reduce the dosage and/or frequency of pharmaceutical agent required, thereby reducing unwanted side effects caused by the pharmaceutical agent. In some embodiments, the KMCT comprise a mixture of capric and caprylic triglycerides. In some embodiments, the KMCT are enriched or purified from coconut oil or palm oil. In some embodiments, the KMCT comprise a mixture of capric and caprylic triglycerides and coconut oil or palm oil. In some embodiments, the total KMCT provided to the migraineur is from about 3 g to about 30 g/day. In some embodiments, the total KMCT provided to the migraineur is from about 10 g to about 30 g/day. Pharmaceutical agents useful in this aspect of the invention include, without limitation, one or more of aspirin, acetominaphen, non-steroidal anti-inflammatory drugs (NSAIDs, such as ibuprofen and naproxofen), triptans (such as sumatriptan (Imitrex), rizatriptan (Maxalt), almotriptan (Axert), naratriptan (Amerge), zolmitriptan (Zomig), frovatriptan (Frova) and eletriptan (Relpax)), ergotamines (such as Ergotamine and Dihydroergotamine), opioid medications, glucocorticoids (such as prednisone and dexamethasone), eptinezumab (Vyepti), erenumab (Aimoving), fremanezumab (Ajovy) and galcanezumab (Emgality).

In another further embodiment, the invention provides a method for augmenting the effects of non-pharmaceutical intervention to reduce the frequency of migraine onset in a migraineur. The method according to this aspect of the invention comprises providing the migraineur with dietary KMCT in combination with a non-pharmaceutical intervention or agent. In some embodiments, the KMCT comprise a mixture of capric and caprylic triglycerides. In some embodiments, the KMCT are enriched or purified from coconut oil or palm oil. In some embodiments, the KMCT comprise a mixture of capric and caprylic triglycerides and coconut oil or palm oil. In some embodiments, the total KMCT provided to the migraineur is from about 3 g to about 30 g/day. In some embodiments, the total KMCT provided to the migraineur is from about 3 g to about 15 g/day. Non-pharmaceutical interventions and agents include, without limitation, one or more of transcutaneous electrical nerve stimulation, transcranial magnetic stimulation, biofeedback, acupuncture, cognitive behavior therapy, ketogenic dieting, butterbur extracts, feverfew, L-cysteine, riboflavin, coenzyme-Q supplements, magnesium supplements, tocopherols (vitamin E), calciferols (vitamin D), ascorbic acids and omega fatty acids.

In yet another embodiment, the invention provides for a method for augmenting the effects of non-pharmaceutical intervention to reduce migraine symptoms experienced by a migraineur. The method according to this aspect of the invention comprises providing the migraineur with dietary KMCT in combination with a non-pharmaceutical intervention or agent. In some embodiments, the KMCT comprise a mixture of capric and caprylic triglycerides. In some embodiments, the KMCT are enriched or purified from coconut oil or palm oil. In some embodiments, the KMCT comprise a mixture of capric and caprylic triglycerides and coconut oil or palm oil. In some embodiments, the total KMCT provided to the migraineur is from about 3 g to about 30 g/day. In some embodiments, the total KMCT provided to the migraineur is from about 10 g to about 30 g/day. Non-pharmaceutical interventions and agents include, without limitation, one or more of caffeine, melatonin, magnesium, feverfew, butterbur, willow extract, ginger, Valerian, coriander seed, dong quai root, rosemary, linden, honeysuckle, mullien, yarrow, wintergreen evadia, tocopherols (vitamin E), calciferols (vitamin D), ascorbic acids and omega fatty acids.

In still yet another embodiment, the invention provides for a method for preventing or reducing the frequency of onset of migraine, and/or reducing migraine symptoms, comprising providing a migraineur with dietary ketone esters, ketone salts or other sources of ketone bodies. The ketone esters or ketone salts are provided in an amount to raise plasma levels of ketone bodies (acetoacetate and [3-hydroxybutyrate) to from about 2 mM to about 5 mM. Preferred ketone esters and ketone salts include, without limitation, [3-hydroxybutyrate-1,3-butanediol monoester, glyceryl-tris-3-hydroxybutyrate, R-3-hydroxybutyrate-R-1,3-butanediol monoester and D-[3-hydroxybutyrate-(R)-1,3-butanediol. Oral administration of such compounds and determination of plasma levels of resulting ketone bodies for other purposes have previously been described. See e.g., Hashim and Vanitallie, J. Lipid Res. 55: 1818-1826 (2014) and Kashiwaya et al., J. Biol. Chem. 285: 25950-29956 (2010).

In another embodiment, the invention provides for a method for preventing or reducing the frequency of onset of migraine, and/or reducing migraine symptoms, comprising providing a migraineur with forms of Vitamin B3. The complex referred to as vitamin B3 comprises niacin, niacinamide and nicotinamide riboside (NR). These forms all produce the compound, nicotinamide adenine dinucleotide (NAD), that is critical for human metabolism, cellular function, transfer reactions and DNA repair. NR is particularly effective for this purpose. NAD is a requisite in enabling cells to produce energy in the mitochondria and works synergistically with other energy sources such as ketones. It is also an important substrate for NAD-consuming enzymes including the sirtuins, that have potential beneficial effects on aging and circadian rhythms, and poly ADP-ribose polymerases, a family of proteins involved in essential cellular functions including genomic stability, DNA repair and programmed cell death, or apoptosis.

Extensive research studies show that NAD levels decrease with advancing age and under metabolic stress. Supplementation of NR has been demonstrated to help support many aspects of healthy aging, including cardiovascular and brain health. It also helps generate energy in mitochondrial-dense tissues like muscle, brain, and liver. NR has achieved GRAS (generally recognized as safe) status in the United States as a food ingredient in nutritional products. The present invention includes NR as an optional adjunctive ingredient for further energy and performance support. Among others, one available credible product to provide the necessary NAD effect has been found in Tru Niagen™ (Chromadex Corporation).

In another embodiment, the invention provides for a method for preventing or reducing the frequency of onset of migraine, and/or reducing migraine symptoms, comprising providing a migraineur with Nicotinamide riboside (NR). Nicotinamide riboside is a unique member of the vitamin B3 family, that functions as a precursor to nicotinamide adenine dinucleotide or NAD+. Cells can use NR to create nicotinamide adenine dinucleotide (NAD+), which is essential to the function of all living cells.

NAD+ is a requisite in enabling cells to produce energy in the mitochondria and in conjunction with an energy source such as ketones operates in a synergistic manner. NAD+ assists in the health of mitochondria and is essential to cellular repair. It is also an important substrate for many NAD+-consuming enzymes including the sirtuins, with potential beneficial effects on aging and circadian rhythms; and PARPs (Poly (ADP-ribose) polymerase) a family of proteins involved in essential cellular functions including genomic stability, DNA repair and apoptosis (programmed cell death).

Extensive research studies show that NAD+ decreases in advancing age over time and under metabolic stress. Supplementation of NR has been demonstrated to help support many aspects of healthy aging, including cardiovascular and brain health. It also helps generate energy in mitochondrial-dense tissues like muscle, brain, and liver.

In 2016 the FDA granted generally recognized as safe (GRAS) status for nicotinamide riboside as a food ingredient in nutritional products. The present inventers intend to incorporate Nicotinamide Riboside in the adjunctive package of ingredients of the present invention to produce as “complete” a system of energy supportive, generative and associated ingredients as possible. Among others, one option for an ideal product to provide the necessary NAD+ effect has been found in Tru Niagen™ (Chromadex Corporation).

In another embodiment, the invention provides for a method for preventing or reducing the frequency of onset of migraine, and/or reducing migraine symptoms, comprising providing a migraineur with a phospholipid. One example of such a phosphor lipid is Phosphatidylserine (PS). Phosphatidylserine is required for healthy nerve cell membranes and myelin. Aging of the human brain is associated with biochemical alterations and structural deterioration that impair neurotransmission. Exogenous PS (300-800 mg/d) is absorbed efficiently in humans, crosses the blood-brain barrier, and safely slows, halts, or reverses biochemical alterations and structural deterioration in nerve cells. It supports human cognitive functions, including the formation of short-term memory, the consolidation of long-term memory, the ability to create new memories, the ability to retrieve memories, the ability to learn and recall information, the ability to focus attention and concentrate, the ability to reason and solve problems, language skills, and the ability to communicate. It also supports locomotor functions, especially rapid reactions and reflexes.

Phosphatidylserine is a phospholipid which has been claimed to enhance neuronal membrane function, and can be derived from several sources. Earlier studies used brain cortex derived PS, of which the human tolerability of 300 mg daily in 130 patients has been shown. The human tolerability of PS derived from soybean has not been reported, although it is widely sold as a nutritional supplement which may improve cognitive function in the elderly. In one study of the safety of two dosages of soy-phosphatidylserine (S-PS) in elderly were reported. Subjects were 120 elderly of both sexes who fulfilled the more stringent criteria for age-associated memory impairment; some also fulfilled the criteria for age-associated cognitive decline. Subjects were allocated at random to one of the three treatment groups: placebo, 300 or 600 mg S-PS daily. Standard biochemical and hematological safety parameters, blood pressure, heart rate and adverse events were assessed at baseline, after 6 and 12 weeks of treatment. No significant differences were found in any of the outcome variables between the treatment groups after Bonferonni-Holme correction. In conclusion, soy derived PS is a safe nutritional supplement for older persons if taken up to a dosage of 200 mg three times daily.

For purposes of the invention, the term “in combination with” means administration in any order, including simultaneous administration, as well as temporally spaced order from a few seconds up to several days apart.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. A composition comprising at least one dietary precursor, at least one phospholipid, and at least one mineral, said composition is designed to reduce migraine symptoms in a patient experiencing migraine headaches.

2. The composition of claim 1 wherein said dietary precursor is a ketone precursor which is ketogenic MCT.

3. The composition of claim 1 wherein said phospholipid is selected from a group consisting essentially of phosphatidylserine, phosphatidylcholine, phosphatidylinositol, phosphotidylethanolamine, phosphatidylglycerol and combinations thereof.

4. The composition of claim 1 wherein said at least one mineral is selected from a group comprising magnesium, calcium, zinc, phosphorus, potassium, chloride, iron, zinc, iodine, sulfur, cobalt, fluoride, manganese, selenium, chromium, and mixtures or combinations thereof.

5. The composition of claim 1 further comprising additives, colorant, flavorings and a carrier.

6. The composition of claim 5 wherein said carrier is selected from a group consisting essentially of gel, powder, liquid, topical, tablets, gel caps, sprays, time release mechanism, pill, boosters, intravenous and mixtures and combinations thereof.

7. A composition comprising at least one dietary precursor, at least one phospholipid, at least one mineral, and at least one dietary ketone, said composition is designed to reduce migraine symptoms in a patient experiencing migraine headaches.

8. The composition of claim 7 wherein said dietary precursor is a ketone precursor which is ketogenic MCT.

9. The composition of claim 7 wherein said phospholipid is selected from a group consisting essentially of phosphatidylserine, phosphatidylcholine, phosphatidylinositol, phosphotidylethanolamine, phosphatidylglycerol and combinations thereof.

10. The composition of claim 7 wherein said at least one mineral is selected from a group comprising magnesium, calcium, zinc, phosphorus, potassium, chloride, iron, zinc, iodine, sulfur, cobalt, fluoride, manganese, selenium, chromium, and mixtures or combinations thereof.

11. The composition of claim 7 wherein dietary ketone is selected from a group consisting essentially of ketones, ketone esters, ketone salts and combinations thereof.

12. The composition of claim 7 wherein said dietary ketone is selected from a group consisting essentially of 3-hydroxybutyrate-1, 3-butanediol monoester, glyceryl-tris-3-hydroxybutyrate, R-3-hydroxybutyrate-R-1, 3-butanediol monoester and D-13-hydroxybutyrate-(R)-1,3-butanediol and mixtures and combinations thereof.

13. The composition of claim 7 wherein said dietary ketone is given to a patient in an amount sufficient to raise plasma ketone bodies to a level from about 0.5 mM to about 6 mM.

14. The composition of claim 7 wherein said phospholipid is given to a patient in an amount from about 25 mg to about 2,000 mg.

15. The composition of claim 7 further comprising at least one Vitamin B.

16. The composition of claim 15 wherein said at least one Vitamin B is selected from a group comprising nicotinamide, niacinamide, niacin, nicotinic acid and mixtures or combinations thereof.

17. The composition of claim 7 further comprising additives, colorant, flavoring and a carrier.

18. The composition of claim 5 wherein said carrier is selected from a group consisting essentially of gel, powder, liquid, topical, tablets, gel caps, sprays, time release mechanism, pill, boosters, intravenous delivery system and mixtures and combinations thereof.

19. A method of manufacturing a composition for treatment of symptoms of migraines, said method comprises admixing at least one dietary precursor, at least one phospholipid, at least one mineral, additives, colorant, and flavoring with an carrier to form a composition designed to reduce migraine symptoms in a patient experiencing migraine headaches.

20. The method of claim 19 further comprising admixing at least one ketone and at least one Vitamin B.
